(19) United States
(12) Patent Application Publication
(10) Pub. No.: US 2011/0263046 A1
(43) Pub. Date: Oct. 27, 2011

(54) Methods of identification of novel ligands for modulation of orphan nuclear receptor RAR-related orphan receptor-gamma (NR1F3) activity

(75) Inventors: Ulrich Deuschle, Worms (DE); Ulrich Abel, Heidelberg (DE); Claus Kremoser, Heidelberg (DE); Thomas Schlueter, Heidelberg (DE); Thomas Hoffmann, Viernheim (DE); Sanja Perovic-Ottstadt, Ginsheim-Gustavsburg (DE)

(73) Assignee: PHENEX PHARMACEUTICALS AG, Ludwigshafen (DE)

(21) Appl. No.: 13/124,860

(22) PCT Filed: Oct. 28, 2009
(86) PCT No.: PCT/EP2009/007717

§ 371 Date: Jul. 14, 2011

Related U.S. Application Data
(60) Provisional application No. 61/108,990, filed on Oct. 28, 2008.

(30) Foreign Application Priority Data
Oct. 28, 2008 (EP) 08018795.8

(51) Int. Cl. G01N 33/566

(52) U.S. Cl. 436/501

(57) ABSTRACT

The invention relates to modulators for the orphan nuclear receptor RORgamma and methods for identification and screening of novel modulators for RORgamma activity as well as methods for treating RORgamma mediated diseases with novel RORgamma modulators identified by such methods.

The invention provides modulators for the orphan nuclear receptor RORgamma and methods for identification and screening of novel modulators for RORgamma activity and methods for treating RORgamma mediated diseases with novel RORgamma modulators identified by such methods.

The retinoid-receptor related orphan receptors consists of three family members, namely RORα (Becker-Andree, Biochem. Biophys. Res. Commun. 1993, 194:1371-1379), RORβ (Andre et al., Gene 1998, 516:277-283) and RORγ (He et al, Immunity 1998, 9:797-806) and constitute the NR1F (ROR/RZR) subgroup of the nuclear receptor superfamily (Mangelsdorf et al., Cell 1995, 83:835-839).

The nuclear receptor superfamily shares common modular structural domains consisting of a hypervariable N-terminal domain, a conserved DNA binding domain (DBD), a hinge region, and a conserved ligand-binding domain (LBD). The DBD targets the receptor to specific DNA sequences (nuclear hormone response elements or NRE's), and the LBD functions in the recognition of endogenous or exogeneous chemical ligands. A constitutive transcriptional activation domain is found at the N-terminus (AF1) and a ligand regulated transcriptional activation domain is embedded within the C-terminal LBD of typical NR's. The nuclear receptors can exist in an transcriptional activating or repressing state when bound to their target NRE's. The basic mechanism of gene activation involves ligand dependent exchange of co-regulatory proteins, namely co-activators and co-repressors (McKenna et al., Endocrine Rev. 1999, 20:321-344). A NR in the repressing state is bound to its DNA recognition element and is associated with co-repressor proteins that recruit histonme-deacetylases (HDACS). In the presence of an agonist, co-repressors are exchanged for coactivators that recruit transcription factors which contribute to assembling of a chromatin-remodelling complex which via histone acetylation relieves transcriptional repression and stimulates transcriptional initiation. The AF-2 domain of the LBD acts as a ligand dependant molecular switch presenting interaction surfaces for co-repressor or co-activator proteins and providing with a conserved mechanism for gene activation or repression that is shared by the members of the nuclear receptor superfamily.

The members of the NR1 F family of nuclear receptors (such as RORgamma) are considered as constitutive active transcription factors in absence of known ligand, which is similar to the estrogen-related receptor alpha (Vanacker et al., Mol. Endocrinol. 1999, 13:764-773). For ERRalpha, synthetic inverse agonists have been described that reduce ERRalpha transcriptional activity by interfering with ERRalpha/PGC1alpha signaling (Willy et al., PNAS 2004, 101:8912-8917). It can be expected that inverse agonists of RORgamma, for instance, should reduce the transcriptional activity of RORgamma and in a functional negative way influence the biological pathways controlled by RORgamma.

The RORs are expressed as isoforms arising from differential splicing or alternative transcriptional start sites. So far, isoforms have been described that differ only in their N-terminal domain (A/B-domain). In humans, four different RORα isoforms have been identified (RORα1-4) while only two isoforms are known for both RORβ (1 and 2) and RORγ (1 and 2) (Andre et al., Gene 1998, 216:277-283; Villey et al., Eur. J. Immunol. 1999, 29:4072-4080). RORgamma is used as a term describing both RORγ1 and/or RORγ2.

The ROR isoforms show different tissue expression patterns and regulate different target genes and physiological pathways. For example, the RORγ2 (also called RORγ-t) is highly restricted to CD4⁺CD8⁺ thymocytes while other tissues express RORγ1 (Eberl et al., Science 2004, 305:248-251).

RORs exhibit a structural architecture that is typical of nuclear receptors. RORs contain four major functional domains: an amino-terminal (A/B) domain, a DNA-binding domain (DBD), a hinge domain, and a ligand-binding domain (LBD) (Evans et al., Science 1988, 240:889-895). The DBD consists of two highly-conserved zinc finger motifs involved in the recognition of ROR response elements (ROREs) which consist of the consensus motif AGGTCA preceded by an AT-rich sequence (Andre et al., Gene 1998, 216:277-283) which is similar to that of the nuclear receptors Rev-ErbAα and Rev-Erbβ (NR1D1 and D2, respectively) (Giguere et al., Genomics 1995, 28:596-598). These recognition elements do also show high similarity to those identified for the estrogen related receptors and in particular ERRα (ERRs, NR3B1, −2, −3) (Vanacker et al., Mol. Endocrinol. 1999, 13:764-773), steroidogenic factor 1 (SF-1, NR5A) and NGFI-B (NR4A1, −2, −3) (Wilson et al., Mol. Cell Biol. 1993, 13:5794-5804).

The Rev-Erb receptors act as constitutive transcriptional repressors, and since they bind to similar DNA recognition sequences, they are able to inhibit ROR-mediated transcriptional activation by competing with RORs for the very same DNA response element (Forman et al., Mol. Endocrinol. 1994, 8:1253-1261). A physiological significance of such an interplay is evident in the control of circadian rhythm, where Rev-Erb and RORα, do repress and activate transcription of the Bmal1 transcription factor, respectively, that plays an important role in the control of the circadian clock (Akashi and Takumi, Nat. Struct. Mol. Biol. 2005, 12:441-448). Such cross-talks between a family member of the RORs and other nuclear receptors binding to similar recognition elements like the ERRα may operate in the control of other physiological pathways as well.

RORα is highly expressed in different brain regions and most highly in cerebellum and thalamus. RORα knock-out mice show ataxia with strong cerebellar atrophy which is highly similar to the symptoms displayed in the so-called staggerer mutant mouse (RORα^{sg/sg}) which carries mutations in RORα that result in a truncated RORα which does not contain a LBD (Hamilton et al., Nature 1996, 379:736-739).

Analysis of RORα^{sg/sg} staggerer-mice have in addition revealed a strong impact on lipid metabolism, namely significant decreases in serum and liver triglyceride, reduced serum HDL cholesterol levels and reduced adiposity. SREBP1c and the cholesterol transporters ABCA1 and ABCG1 are reduced in livers of staggerer mice and CHIP analysis suggest that RORα is directly recruited to and regulates the SREBP1c promoter. In addition, PGC1α, PGC1β, lipin1 and β2-adrenergic receptor were found to be increased in tissues such as liver or white and brown adipose tissue, which may help to explain the observed resistance to diet-induced obesity in staggerer mice (Lau et al., J. Biol. Chem. 2008, 283:18411-18421).

RORβ is more differentially expressed, namely in certain regions of the brain and in the retina. RORβ knock-out mice display a duck-like gait and retinal degeneration which leads to blindness (Andre et al., EMBO. J. 1998, 17:3867-3877). The molecular mechanisms behind this retinal degeneration are still poorly understood.

RORγ (particularly RORγ2) null-mutant mice lack lymph nodes and Peyer's patches (Eberl and Littmann, Immunol. Rev. 2003, 195:81-90) and lymphatic tissue inducer (LTi) cells are completely absent from spleen mesentery and intestine. In addition, the size of the thymus and the number of thymocytes is greatly reduced in RORγ null mice (Sun et al., Science 2000, 288:2369-2373) due to a reduction in double-positive CD4⁺CD8⁺ and single positive CD4⁻CD8⁺ or CD4⁺CD8⁻ cells suggesting a very important role of RORγ2 in thymocyte development.

Thymocyte development follows a complex program involving coordinated cycles of proliferation, differentiation, cell death and gene recombination in cell populations dedicated by their microenvironment. Pluripotent lymphocyte progenitors migrating from fetal liver or adult bone marrow to the thymus are being committed to the T cell lineage. They develop through a series of steps from CD4⁻CD8⁻ double negative cells to CD4⁺CD8⁺ cells and those with low affinity towards self MHC peptides are eliminated by negative selection. These develop into CD4⁻CD8⁺ (killer) or CD4⁺CD8− (helper) T-cell lineages. RORγ2 is not expressed in double negative and little expressed in immature single negative thymocytes (He et al., J. Immunol. 2000, 164:5668-5674), while highly upregulated in double-positive thymocytes and downregulated during differentiation in single-positive thymocytes. RORγ deficiency results in increased apoptosis in CD4⁺CD8⁺ cells and the number of peripheral blood thymocytes is decreased by 6-fold (10-fold CD4⁺ and 3-fold CD8⁺ thymocytes).

Recent experiments in a model of ovalbumin (OVA)-induced inflammation in mice, as a model for allergic airway disease, demonstrated a severe impairment of the development of the allergic phenotype in the RORγ KO mice with decreased numbers of CD4⁺ cells and lower Th2 cytokine/chemokine protein and mRNA expression in the lungs after challenge with OVA (Tilley et al., J. Immunol. 2007, 178:3208-3218). IFN-γ and IL-10 production were increased in splenocytes following re-stimulation with the OVA antigen compared to wt splenocytes suggesting a shift towards a Th1 type immune response on cost of a reduction of Th2 type response. This suggests that down-modulation of RORγ transcriptional activity with a ligand could result in a similar shift of the immune response towards a Th2 type response which could be beneficial in the treatment of certain allergic inflammatory conditions.

T-helper cells were previously considered to consist of Th1 and Th2 cells. However, a new class of Th cells, the Th17 cells which produce IL-17, were identified as a unique class of T helper cells that are considered to be pro-inflammatory. They are recognized as key players in autoimmune and inflammatory diseases since IL-17 expression has been associated with many inflammatory diseases of potential autoimmune etiology such as Multiple Sclerosis, Rheumatoid Arthritis, Systemic Lupus Erythematosus and inflammatory bowel disease such as Crohn's Disease or Colitis Ulcerosa (Ivanov et al., Cell 2006, 126:1121-1133; Tesmer et al., Immunol. Rev. 2008, 223:87-113). Another disease with a strong autoimmune etiology component is Type 1 Diabetes. Recently, a link between an increased activity of Th17 cells and Type 1 Diabetes was drawn (Bradshaw et al., J. Immunol. 2009 183:4432-4439; Emamaullee et al., Diabetes. 2009 58:1302-1311). Inflammatory skin diseases with an autoimmune component such as psoriasis, neurodermitis and atopic eczema are also believed to be associated with an increased activity of Th17 cells (Miossec, Microbes Infect. 2009, 11:625-630).

RORγ2 is exclusively expressed in cells of the immune system and has been identified as a master regulator of Th17 cell differentiation. Expression of RORγ2 is induced by TGF-beta or IL-6 and overexpression of RORγ2 results in increased Th17 cell lineage and IL-17 expression. RORγ2 KO mice show very little Th17 cells in the intestinal lamina propria but Th17 cells are still detectable. Recently Yang et al. (2008) reported the expression of RORα in Th17 cells which is regulated by TGF-beta and IL-6 in a STAT3 dependent fashion. Double mutations in RORα and RORγ2 completely inhibited Th17 differentiation in vitro and in vivo and completely blocked the development of symptoms in a model of EAE (experimental autoimmune encephalitis) (Yang et al., Immunity 2008, 28:29-39). This suggests that RORγ2 and RORα synergistically control Th17 development. Inhibitors of both RORγ2 and RORα may inhibit the development of Th17 cells and the expression of pro-inflammatory IL-17 in inflammatory diseases. Inhibition of IL-17 production via inhibition of Th17 cell development may also be advantageous in atopic dermatitis and psoriasis where IL-17 is deeply involved. Interestingly, recent evidence was presented that IL-10 suppresses the expression of IL-17 secreted by both macrophages and T cells. In addition, the expression of the Th17 transcription factor RORγ2 was suppressed (Gu et al., Eur. J. Immunol. 2008, 38:1807-1813). Moreover, IL-10 deficient mice provide a good model for Inflammatory Bowel Disease (IBD) where a shift towards a Th1 type inflammatory response is frequently observed. Oral IL-10 delivery poses a potential treatment option for IBD.

RORγ1 is expressed in muscle and several other tissues including pancreas, thymus, prostate, liver and testis. Ectopic overexpression of dominant active and dominant negative versions of RORγ1 showed that this receptor controls genes involved in lipid metabolism (FABP4, CD36, LPL and UCP3), cholesterol efflux (ABCA1, ABCG1) (carbohydrate metabolism (GLUT5, adiponectin receptor 2 and IL-15) and muscle mass (myostatin and IL-15).

RORα1 and RORγ1 are expressed in liver and oscillate in a circadian fashion. Double KO mice show that these genes are involved in regulation of phase I and phase II metabolic enzymes including 3-beta-hydroxysteroid dehydrogenases, Cyp450 enzymes and sulfotransferases, suggesting important roles in steroid, bile acid and xenobiotic metabolisms (Kang et al., Physiol. Genomics 2007, 31:281-294). One of the genes regulated was shown to be Cyp7b1 which has an important role in cholesterol metabolism and it was shown that RORα is necessary and sufficient for regulation of Cyp7b1. Analysis of target genes in RORα and LXRa KO mice raised the hypothesis that both receptors are mutually suppressive with respective to their target genes (Wada et al., Exp. Biol. Med. 2008, 233:1191-1201).

### Ligands for the RORs:

It was reported that cholesterol and its sulfated derivatives might function as RORα ligands and in particular cholesterol-sulfate could restore transcriptional activity of RORα in cholesterol depleted cells (Kallen et al., Structure 2002, 10:1697-1707). Previously, melatonin (Missbach et al., J. Biol. Chem. 1998, 271:13515-13522) and thiazolidindiones were suggested to bind to RORα (Wiesenberg et al., Nucleic Acid Res. 1995, 23:327-333). However, none of these have been shown to be functional ligands of RORα or any of the other RORs. Certain retinoids including all-trans retinoid acid have been demonstrated to bind to RORβ and function as partial antagonists for RORβ but not RORα (Stehlin-Gaon et al., Nat. Struct. Biol. 2003, 10:820-825). However, none of these or any other ligands have been described yet to bind to and/or modulate the transcriptional activity of RORγ1 or RORγ2 despite the cloning of the mouse and human RORgamma cDNAs as a potential basis for generating methods for screening of substance libraries for RORgamma modulating compounds (Medvedev et al., Gene 1996, 181:199-206, WO 2000/24757).

It is therefore the object of the present invention to provide compounds which bind to the orphan nuclear receptors RORγ1 and/or RORγ2 and, thus, to open new methods for treating diseases associated with the modulation of RORgamma, such as autoimmune diseases, inflammatory skin diseases or multiple sclerosis.

It is further the object of the present invention to provide a screening method for identifying the ligands of RORgamma and for measuring the activity of said ligands.

This object is solved by the surprising discovery of small molecule ligands for the human RORgamma. Among these ligands are retinoids, but also rexinoids.

Thus, the present invention provides RORgamma modulators which can be used for treating or preventing a disease or disorder associated with the inactivation or activation of the RORgamma receptor.

The present invention further provides a method for modulating RORgamma activity in a cell culture or in a biochemical cell-free in vitro assay system comprising the step of administrating to such cell culture or assay system an effective amount of a RORgamma modulator as described herein, sufficient to induce or reduce the readout of RORgamma activity in such cell culture or biochemical assay system.

Furthermore, the present invention relates to compounds identified by the methods described herein.

### BRIEF DESCRIPTION OF THE FIGURES

FIG. 1 shows the results of a radioligand displacement assay, wherein the replacement of radioactive H³-25-hydroxycholesterol, bound to the RORgamma-ligand binding domain (RORγ-LBD), by the compounds described herein is measured.

FIG. 2 shows the structural formulas of the compounds used in the present invention.

FIG. 3 shows the reduction of the level of IL-17 by the compound LE135 in peripheral blood human mononuclear cells (PBMCs) in a dose-response manner.

The present invention relates to a RORgamma modulator for use in the treatment or prophylaxis of a disease or disorder associated with the inhibition or activation of a RORgamma receptor.

The present invention also relates to the use of a RORgamma modulator for the preparation of a medicament for treating or preventing a disease or disorder associated with the inhibition or activation of a RORgamma receptor.

The present invention also relates to a method of treating or preventing a disease or disorder associated with the inhibition or activation of a RORgamma receptor, comprising administering to a subject in need of such treatment an effective amount of a RORgamma modulator.

When treating the disease or disorder associated with the modulation of the RORgamma receptor, the activity of said receptor is preferably reduced.

Preferably, the disease or disorder is selected from the group of diseases with Th17 associated tissue inflammation consisting of autoimmune diseases, inflammatory skin diseases and multiple sclerosis.

The RORgamma modulator used in the present invention preferably comprises a compound of formula (I) with the following structure

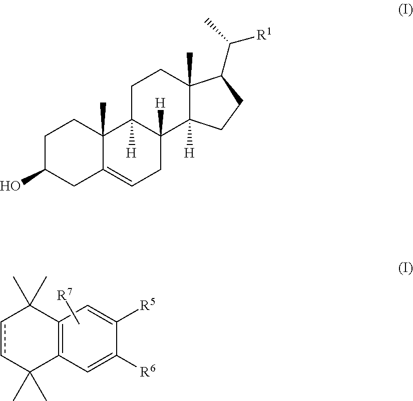

or a solvate or a pharmaceutically acceptable salt thereof, wherein

R⁵ is CONHR⁸, NHCOR⁸, C(O)R⁸, CH═CHR⁸, C(CH₃)═CHR⁸, C═CR⁸, CH(OH)CH═CHR⁸, C(O)CH═CHR⁸, 5 to 6 membered heterocyclyl-R⁸,

R⁶ is hydrogen,

R⁵ and R⁶ may also together form

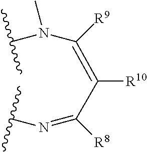

R⁷ is hydrogen, fluorine, chlorine or hydroxy,

R⁸ is 4-yl-benzoic acid or 6-yl-2-naphthoic acid, and

R⁹ and R¹⁰ are hydrogen or R⁹ and R¹⁰ form together with the bond to which they attach a fused 5-10 membered heteroaromatic or aromatic monocyclic or bicyclic ring.

In the above and the following, the employed terms have independently the meaning as described below:

A 5 to 10 membered aromatic mono- or bicyclic moiety is preferably selected from phenyl, biphenyl, naphthyl, tetrahydronaphthyl, fluorenyl, indenyl and phenanthrenyl, more preferably phenyl and naphthyl.

A 5 to 10 membered heteroaromatic monocyclic or bicyclic ring is a ringsystem having 4 to 9 carbon atoms and at least one ring containing at least one heteroatom selected from O, N and/or S. Preferably, heteroaryl contains 1, 2, 3 or 4, more preferably 1, 2 or 3 heteroatoms selected from O and/or N and is preferably selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl and furopyridinyl. Spiro moieties are also included within the scope of this definition. Preferred heteroaryl includes pyridinyl, imidazolyl, pyrimidinyl, pyrazolyl, triazolyl, pyrazinyl, tetrazolyl, isoxazolyl, oxazolyl, isothiazolyl, oxadiazolyl and triazolyl.

Heterocyclyl is a 5 to 6 membered saturated or unsaturated ring containing at least one heteroatom selected from O, N and/or S and 1, 2, 3, 4, or 5 carbon atoms. Preferably, heterocyclyl contains 1, 2, 3 or 4, more preferably 1, 2 or 3 heteroatoms selected from O and/or N. Heterocyclyl includes mono- and bicyclic ringsystems and is preferably selected from pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, azetidin-2-one-1-yl, pyrrolidin-2-one-1-yl, piperid-2-one-1-yl, azepan-2-one-1-yl, 3-azabicyco[3.1.0] hexanyl, 3-azabicyclo[4.1.0]heptanyl, azabicyclo[2.2.2]hexanyl, 3H-indolyl and quinolizinyl. Spiromoieties are also included within the scope of this definition.

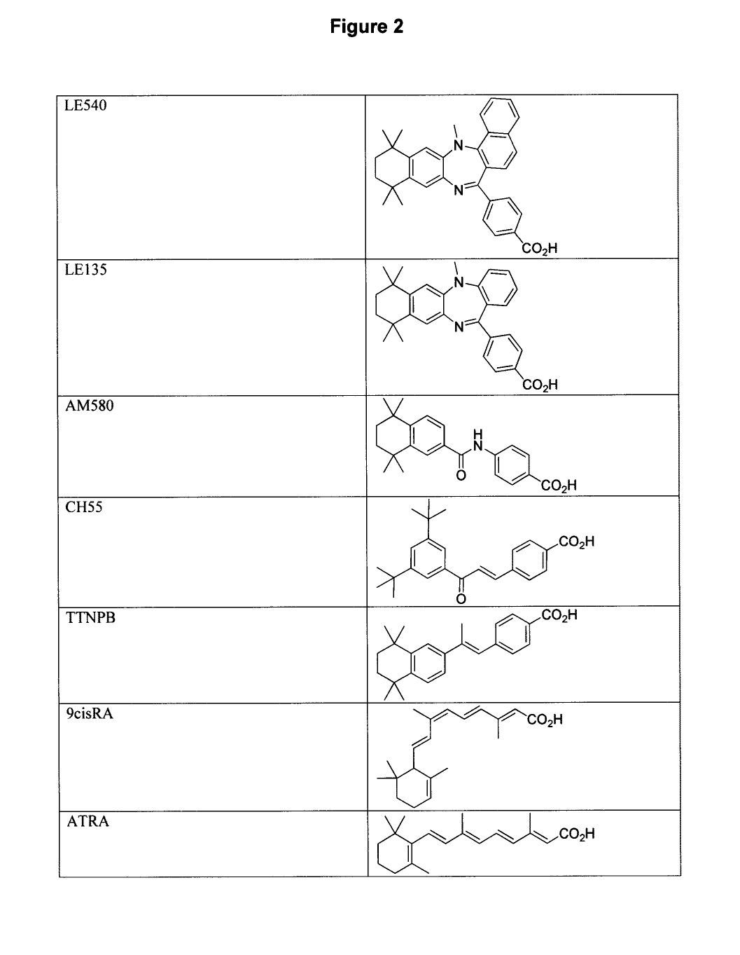

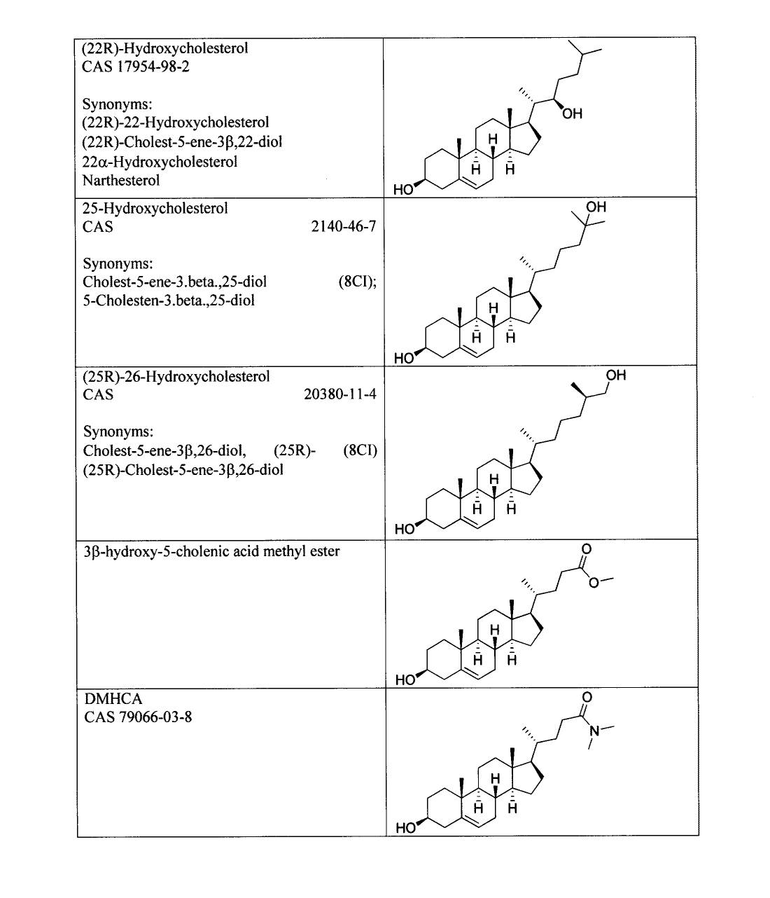

Preferred embodiments of the compounds used in the present invention are shown in FIG. 2.

The compounds used in the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present on these groups and can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e. groups which can be protonated, can be present and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

In practical use, the compounds used in the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the composition for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The compounds used in the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

When treating or preventing RORgamma mediated conditions for which compounds of formula (I) are indicated, generally satisfactory results are obtained when the compounds are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1 milligram to about 1,000 milligrams, preferably from about 1 milligram to about 50 milligrams. In the case of a 70 kg adult human, the total daily dose will generally be from about 7 milligrams to about 350 milligrams. This dosage regimen may be adjusted to provide the optimal therapeutic response.

For the first time, the present invention describes modulators, in the following also referred to as ligands, which bind to the RORgamma receptor. Surprisingly, it has been found that certain synthetic retinoids such as compounds of formula (I) act as modulators of the RORgamma receptor.

The compounds of formula (I) show antagonistic activity with respect to the dose dependent modulation of the constitutive interaction of the RORγ ligand binding domain with peptides derived from the coactivators such as SRC-1 or TIF-2.

It has been surprisingly found that the interaction between RORγ ligand binding domain and the peptides can be determined by a homogenous FRET based ligand sensing assays.

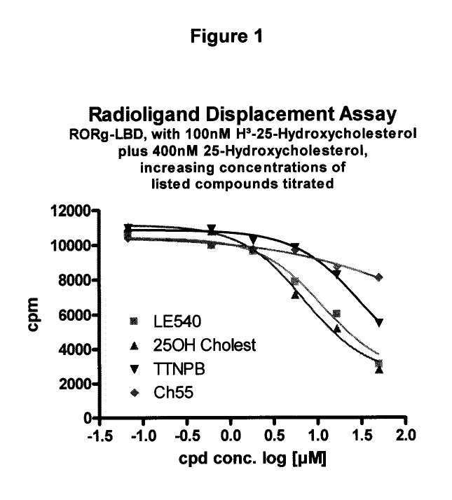

In a specific radioactive displacement assay using ³H-25-hydroxycholesterol at saturating concentration (500 nM), a dose-dependent competitive displacement by non labelled 25-hydroxycholesterol, LE540, TTNPB or CH55 took place (see FIG. 1).

The identification of high affinity ligands for RORγ with receptor activity modulating properties is the basis to enable experts knowledgeable in the field to establish assays for the identification of novel agonistic and antagonistic RORγ ligands from libraries of small molecules. The identification of ligands which bind to and modulate the activity of RORγ1 and RORγ2 is the first mandatory step to develop new small molecule based medicines with a potential to be developed for the treatment of diseases which are directly or indirectly controlled by the activity of RORγ1 or RORγ2. Such diseases include but are not restricted to inflammatory diseases, rheumatoid arthritis, autoimmune diseases or diseases with an autoimmune componente such as Systemic Lupus Erythomatosis, Inflammatory Bowel Disease (Crohn's Disease), Ulcerative Colitis, Type 1 Diabetes, and Inflammatory Skin Diseases such as Atopic Eczema or Psoriasis, Multiple Sclerosis or similar diseases.

Thus, the present invention also relates to a method for identifying novel ligands of the RORgamma receptor.

Different assay systems can be used for the de novo identification of nuclear receptor ligands, in general. Such assay systems are either biochemical, cell-free assays that employ a purified receptor from a natural cell environment, or preferably, a purified recombinant version of the nuclear receptor under investigation, or they are cell-based assay systems. The nuclear receptor under investigation is usually employed either as a full length receptor or as parts thereof that cover at least those amino acid residues that constitute the ligand binding domain of the nuclear receptor. The ligand binding domain (LBD) is defined as that protein domain of a nuclear receptor that extends distal or C-terminally from the highly conserved Zinc-finger containing DNA binding domain and from the less conserved hinge region up to the C-terminus of the nuclear receptor.

For a biochemical assay the nuclear receptor or an LBD-containing part thereof is recombinantly expressed in one of the usual expression systems such as E. coli, yeast, baculovirus-induced insect cells or a mammalian cell culture system. The nuclear receptor expression construct can be either native, i.e. resembling entirely a naturally occurring amino acid sequence or, preferably, it can contain certain artificial amino acid stretches that resemble affinity tags for the ease of purification. Alternatively, the NR construct can be fused to another protein or protein domain that acts as an affinity, or localisation tag or as a folding and stabilization aid.

The recombinantly expressed NR protein is then purified using a standard method which is available for those skilled in the art of protein expression and purification up to a degree which allows characterization of its ligand binding activity. For the purpose of ligand screening, the recombinant NR protein can be used as such or it can be further labeled or decorated with labeling reagents. Unmodified proteins can be used in radioligand or fluorescent ligand displacement assays where a radiolabelled or fluorescently-tagged bona fide ligand is available as a reference ligand. For the use in FRET or Alphascreen® type assays, the recombinant NR protein must be decorated with further reagents such as an Europium-chelate fluorophore containing antibody directed against an affinity tag. Such decoration reagents emit a primary signal that can be further transferred, enhanced or complemented by a second reagent. In case of the FRET assay, the second reagent is a chromophore than can absorb light of the wavelength that is emitted by the primary reagent which is attached to the NR protein. When an Eu-chelate is the primary fluorescent light source originating from the NR-decorated reagent, then i.e. Allophycocyanin (APC) can be the second fluorescent absorber. The Fluorescence Resonance Energy Transfer (FRET) effect only takes place when emitter and absorber come into close proximity.

Therefore, most non radiolabeled biochemical nuclear receptor assays make use of the protein recruiting capabilities of nuclear receptors. Nuclear receptors, in general, tend to recruit certain adaptor proteins that tether them to chromatin and transcriptional activity modifying protein complexes. In the absence of ligand, most NRs recruit so-called corepressors that contain or further recruit Histone Deacetylase activity and hence keep the chromatin region around the NR response element transcriptionally silent. When an activating ligand, an agonist, binds to the nuclear receptor, the corepressors are replaced by co-activators, adaptor proteins that recruit other proteins with Histone-Acetylase activity. The resulting chromating opening can then result in increased transcriptional activity starting from these promoter regions.

Thus, the recruitment of co-activators is a critical step in the activation cascade initiated by an agonist ligand. Therefore biochemical assays system can detect ligand binding by detecting the recruitment of co-activators. This co-activator recruitment, in turn, can be detected if the co-activator is labeled by the secondary reagent required for this assay type. Nuclear Receptor ligands can be identified using a nuclear receptor-peptide interaction assay that utilizes time-resolved fluorescence resonance energy transfer (TR-FRET). This assay is based on the principle finding that ligands can induce conformational alterations upon binding within the ligand binding domain (LBD) of nuclear receptors that alter interactions with coactivator or corepressor proteins which in turn mediate alterations in transcriptional activity. In TR-FRET, a fluorescent donor molecule transfers energy via dipole-dipole interaction to an, usually fluorescent, acceptor molecule. This technique is a standard spectroscopic technique for measuring distances and changes in distances in the 10-70 Å range, which depends on the R⁻⁶ distance between donor and acceptor molecule. Using europium cryptate in conjugation with the multichromophoric Allophycocyanine, interactions with a very large R₀ of 90 Å can be achieved (Mathis et al., Clin. Chem. 1993, 39:1953-1959).

For the purpose of a nuclear receptor ligand sensitive FRET assay, one can attach the acceptor label to one of the known coactivators proteins, preferably to a peptide that is derived from a coactivator. Usually, a 20 to 30mer peptide that resembles one of the well defined LXXLL-motifs that are responsible for the physical NR-cofactor interaction is sufficient. The fluorophore label can be attached to such a peptide by various means, including a biotinylation of the peptide and capture of the biotin by an e.g. streptavidin-APC complex.

In the case of RORgamma, a certain constitutive activity in such biochemical assay systems can be observed. This means just the NR together with cofactor peptides and all necessary reagents creates a signal already. In the case of such constitutive active NRs it can be possible to further stimulate such signal by agonist compounds that further stabilize or enhance the active NR conformation. Compounds, however, that lead to a dose dependent signal decrease in the case of such constitutive active receptors are termed inversed agonists.

For the purpose of reducing the pro-inflammatory Th17 cell count in conjunction with amelioration of RORgamma mediated immunological diseases the identification of such inverse agonists is sought.

For cell-based nuclear receptor assays, mostly recombinant expressed versions of the nuclear receptor under investigation are used that are brought into the cell either by transient transfection or by transfection and subsequent selection into a stably nuclear receptor construct expressing cell line. Such cell lines that transiently or stably express a nuclear receptor or an LBD-containing part thereof can be used for ligand screening when they either harbor a plasmid where said nuclear receptor can initiate the transcription of a certain reporter gene under the control of a nuclear receptor constructs-specific DNA response element in the reporter promoter, or the nuclear receptor constructs which is transfected controls the transcription of a certain native target gene. Changes in endogenous or native target gene expression which are brought about by a certain ligand modulating the NR's transcriptional control over such target genes can be monitored with any target gene mRNA specific detection systems such as quantitative real time polymerase chain reaction (qRT-PCR) based methods like Taqman®, Light Cycler®, Sybr Green® incorporation or similar techniques. Examples for reporter genes whose expression levels can be directly monitored through monitoring the activity of the encoded reporter enzyme are Luciferases, Chloramphenicol-Acetyltransferases or similar well defined reporter enzymes.

An agonist ligand in such a RORgamma cell-based reporter assay would stimulate the constitutive reporter signal further, an inverse agonist ligand would dose dependently reduce the reporter signal.

As described in more detail in the examples, a GST-RORgamma-LBD fusion protein is expressed and purified from E. coli. The TR-FRET assays were performed in a final volume of 25 μl in individual wells of a 384 well plate using a Tris-based buffer system: 10-50 mM Tris-HCl pH 7.9; 50-100 mM KCl, 1-10 mM MgCl₂; 20-100 ng/μl BSA), containing 20-60 ng/well recombinantly expressed RORγ-LBD fused to GST, 200-600 nM N-terminally biotinylated peptide (e.g. derived from SRC-1 or TIF-2), 50-500 ng/well Streptavidin-xIAPC conjugate (Prozyme) and 2-20 ng/well Eu W1024-antiGST (Perkin Elmer). The TR-FRET signal was detected using a Perkin Elmer VICTOR2V™ Multilabel Counter by detecting emitted light at 665 nm and 615 nm and the results plotted as the ratio of 665/615 nm.

It is common understanding to those skilled in the art, that the RORgamma-LBD moiety in the GST fusion protein can be exchanged by RORgamma containing protein fragments which are smaller or larger (e.g. the fullength RORγ1 or RORγ2). Also the GST moiety can be exchanged by other affinity tags (e.g. His-tag, myc-tag, HA-tag) in combination with the respective Europium-labelled antibodies detecting the used affinity tags. In addition, the biotinylated peptide derived from the nuclear receptor interacting domains of coactivator proteins (e.g. SRC-1 and TIF-2) can be exchanged by larger fragments of said coactivators or even fullength coactivators which are recombinantly expressed in prokaryotic of eukaryotic systems and biotinylated in vitro or in vivo. The nuclear receptor-peptide interaction assay described herein could also be performed using alternative detection methodologies such as Fluorescence Polarization (FP (WO 1999/027365) NUCLEAR HORMONE RECEPTOR DRUG SCREENS) and AlphaScreen using a Fusion Alpha Multilabel Reader (commercially available via PerkinElmer). For those skilled in the art, both fluorescently labelled peptides as well as a fluorescently labelled ligand could be used for detection of ligand interaction with RORgamma or ligand mediated interaction of a cofactor derived peptide with RORgamma.

RORgamma is believed to be a key differentiation factor for Th17 cells as well as a direct transcription factor to stimulate transcription of the Interleukin 17 (IL-17) gene in differentiated Th17 cells (Zhang et al., Nat. Immunol. 2008, 9:1297-1306). Thus measuring IL-17 in a cell culture supernatant from T-cells or more generally, leukocytes, might be an appropriate measure to determine the impact of RORgamma and RORgamma modulation on Th17 cell differentiation and their activity. Such leukocytes can be isolated as so-called “buffy coats”, the interface between red blood cells and the plasma supernatant after low-speed centrifugation of human or animal blood. These “buffy coats” contain a mixture of Peripheral Blood Mononuclear Cells (PBMCs) and Thrombocytes and the T-cell part therein can be well stimulated to secrete IL-17. Such a PBMC cell culture is a good system to check for effects of known or potential immunomodulatory and immunosuppressive compounds (Zhang et al., Cytokine. 2008, 42:345-352).

The following examples describe the invention in more detail. These examples, however, are not construed to limit the scope of the invention in any manner.

### EXAMPLES

### Protein Expression and Purification

For determination of a ligand mediated cofactor peptide interaction to quantify ligand interaction with the nuclear receptor Retinoid Acid Receptor-related Orphan Receptor gamma (RORγ), the respective Ligand Binding Domain (LBD) of RORgamma was expressed in E. coli and purified as described below:

The human RORg ligand binding domain (LBD) was expressed in E. coli strain BL21(DE3) as an N-terminal glutathione-S-transferase (GST) tagged fusion protein. The DNA encoding the RORg ligand binding domain was cloned into vector pDEST15 (Invitrogen). The amino acid boundaries of the ligand binding domain were amino acids 267-518 of Database entry NM_{—}005060 (RefSeq). Expression in pDEST15 is controlled by a IPTG inducible T7 promoter and cloning and transformation of E. coli was done essentially according to standard protocols known to persons skilled in the art and supplied by Invitrogen.

Expression and purification of the RORγ-LBD: An overnight preculture of E. coil strain BL21 (DE3) (Invitrogen) transformed with pDEST15-huRORg-LBD was diluted 1:20 in LB-Ampicillin medium and grown at 30° C. to an optical density of OD600=0.6. Gene expression was then induced by addition of IPTG to an end concentration of 0.5 mM. Cells were incubated an additional 16 h at 16° C., 180 rpm. Cells were collected by centrifugation (7,000×g, 10 min, room temperature). Cells were resuspended in 10 ml lysis buffer (50 mM Tris-HCl pH 7.5, 20 mM NaCl, 5 mM EDTA and 4 mg/ml lysozyme) per gram wet pellet weight and left at room temperature for 30 min. Subsequently 1 μl

DNasel solution (2 mg/ml) per ml solution is added and MgCl₂ is added to 20 mM final concentration and the resulting solution is incubated on ice for 15 min. Cells were then subjected to sonication and cell debris removed via centrifugation (14,000×g, 60 min, 4° C.). Per 1 l of original cell culture 0.5 ml prewashed Glutathione 4B sepharose slurry (Pharmacia) was added and the suspension kept slowly rotating for 1 h at 4° C. Glutathione 4B sepharose beads were pelleted by centrifugation (1,000×g, 1 min, 4° C.) and washed three times in wash buffer (25 mM Tris-HCl, pH 7.5, 50 mM KCl, 4 mM MgCl₂ and 1 M NaCl). The pellet was re-suspended in 500 μl elution buffer per gram wet pellet weight (elution buffer: 20 mM Tris-HCl, pH 7.5, 60 mM KCl, 5 mM MgCl₂ and 10 mM glutathione added immediately prior to use as powder). The suspension was left rotating for 15 min at 4° C., the beads pelleted and eluted again with 50 mM glutathione in elution buffer. For subsequent TR FRET assays, glycerol was added to this protein solution to 10% (v/v).

For the radioligand displacement assay (Example 3), the eluate was dialysed overnight in 20 mM Tris-HCl buffer (pH 7.5) containing 60 mM KCl, 5 mM MgCl₂ and used directly in the assay.

Determination of the interaction of a ligand with the human RORgamma ligand binding domain was done by using a Ligand Sensing Assay based on Time-resolved Fluorescence Energy Transfer (TR-FRET).

### TR-FRET Activity Assay

This method measures the ability of putative ligands to modulate the interaction between the purified bacterial expressed RORg ligand binding domain (LBD) and synthetic N-terminally biotinylated peptides which are derived from nuclear receptor coactivator proteins such as but not limited to SRC1 (NcoA1), SRC2 (NcoA2,TIF2), SRC3 (NcoA3), PGC1α, PGC1β, CBP, GRIP1, TRAP220, RIP140. The peptides used for Example 1 and Example 2 are listed in Table 1 below:

TABLE 1

| Peptide Name | DB entry | DB entry | |
|---|---|---|---|
| (aa range) | Protein | DNA | Sequence |
| SRC1(676-700) | NP_003734 | NM_003743 | NH2-CPSSHSSLTERHKILHRLLQEGSPS-COOH |
| TIF2(628-658) | NP_006531 | NM_006540 | NH2-GQSRLHDSKGQTKLLQLLTTKSDQ-COOH |

The ligand binding domain (LBD) of RORγ was expressed as fusion protein with GST in BL-21 (BL3) cells using the vector pDEST15. Cells were lysed by lysozyme-treatment and sonication, and the fusion proteins purified over glutathione sepharose (Pharmacia) according to the manufacturers instructions. For screening of compounds for their influence on the RORγ-peptide interaction, the LANCE technology (Perkin Elmer) was applied. This method relies on the binding dependent energy transfer from a donor to an acceptor fluorophor attached to the binding partner of interest. For ease of handling and reduction of background from compound fluorescence LANCE technology makes use of generic fluorophore labels and time resolved detection assays were done in a final volume of 25 μl in a 384 well plate, in a Tris-based buffer (20 mM Tris-HCl pH 7.9; 60 mM KCl, 5 mM MgCl₂; 35 ng/μl BSA), containing 20-60 ng/well recombinantly expressed RORγ-LBD fused to GST, 200-600 nM N-terminally biotinylated peptide, 200 ng/well Streptavidin-xIAPC conjugate (Prozyme) and 6-10 ng/well Eu W1024-antiGST (Perkin Elmer). DMSO content of the samples was kept at 1%. After generation of the assay mix the potentially RORγ modulating ligands were diluted. After his step, the assay was equilibrated for one hour in the dark at room temperature in FIA-plates black 384 well (Greiner). The LANCE signal was detected by a Perkin Elmer VICTOR2V™ Multilabel Counter. The results were visualized by plotting the ratio between the emitted light at 665 nm and 615 nm. A basal level of RORγ-peptide formation is observed in the absence of added ligand. Ligands that promote the complex formation induce a concentration-dependent increase in time-resolved fluorescent signal. Compounds which bind equally well to both monomeric RORγ and to the RORγ-peptide complex would be expected to give no change in signal, whereas ligands which bind preferentially to the monomeric receptor would be expected to induce a concentration-dependent decrease in the observed signal.

### Example 1

To assess the agonistic and antagonistic potential of the compounds, EC₅₀ or IC₅₀-values were determined using a Ligand Sensing Assay based on Time-resolved Fluorescence Energy Transfer (TR-FRET) as described above. The normalised TR FRET assay values, using the following equation: 1000*655 nm measurement value/615 nm measurement value, were transferred to the program GraphPad Prism to generate graphs and dose response curves using the following equation:

Equation: Sigmoidal dose-response (variable slope) Y=Bottom+(Top-Bottom)/(1+10̂((LogEC50-X)*HillSlope))

X is the logarithm of the concentration. Y is the response

Y starts at Bottom and goes to Top with a sigmoidal shape.

This is identical to the “four parameter logistic equation”. The EC₅₀ or IC₅₀ values are calculated using this equation.

Examples for selected compounds are listed in Table 2 below:

TABLE 2

| Compound | | Peptide | |
|---|---|---|---|
| | | | IC₅₀ [nM] |
| LE540 | antagonistic | SRC1(676-700) | 3600 |
| LE540 | antagonistic | TIF2(628-658) | 4900 |
| LE135 | antagonistic | SRC1(676-700) | 19300 |
| LE135 | antagonistic | TIF2(628-658) | 13200 |
| Am580 | antagonistic | SRC1(676-700) | 23400 |
| Am580 | antagonistic | TIF2(628-658) | 19800 |
| Ch55 | antagonistic | SRC1(676-700) | 12200 |
| Ch55 | antagonistic | TIF2(628-658) | 11300 |
| TTNPB | antagonistic | SRC1(676-700) | 8600 |
| TTNPB | antagonistic | TIF2(628-658) | 8200 |
| 9cisRA | antagonistic | SRC1(676-700) | 14000 |
| 9cisRA | antagonistic | TIF2(628-658) | 15900 |
| ATRA | antagonistic | SRC1(676-700) | 18300 |
| ATRA | antagonistic | TIF2(628-658) | 14200 |
| | | | EC₅₀ [nM] |
| 22R-Hydroxycholesterol | agonistic | TIF2(628-658) | 16.5 |
| 25-Hydroxycholesterol | agonistic | TIF2(628-658) | 18.6 |
| (25R)-26-Hydroxycholesterol | agonistic | TIF2(628-658) | 14.1 |
| Cholenic Acid Methyl Ester | agonistic | TIF2(628-658) | 27.5 |
| DMHCA | agonistic | TIF2(628-658) | 10.7 |

All compounds listed above with retinoid structures (LE540, LE135, Am580, Ch55, TTNPB, 9cisRA and ATRA) do reduce the signal in the TR-FRET assay in a dose dependent fashion with IC₅₀ values ranging from 3,600 nM for LE540 to 23,400 nM for Am580 using SRC1 as interacting peptide.

In contrast, the oxysterol compounds 22R-hydroxycholesterol, 25-hydroxycholesterol, (25R)-26-hydroxycholesterol, cholenic acid methyl ester and DMHCA do act agonistic in a highly potent fashion with EC₅₀ values ranging from 10.7 nM for DMHCA to 27.7 nM for cholenic acid methyl ester with TIF2 as interacting peptide.

### Example 2

In a variation of the assay described above, an antagonistic compound was added in a saturating concentration (4 μM) to the assay mix. Then the agonistic compounds were diluted and the assay was equilibrated for one hour in the dark. Titration of agonists on top of saturating antagonist concentration, still produced dose response curves for the agonists, but with higher apparent EC₅₀-values for the agonists compared to the data obtained in absence of antagonist (see Table 2). This shows that the antagonist can be replaced by the agonists. The apparent EC₅₀-values were as listed below in Table 3:

TABLE 3

| Compound (assay includes | | | |
|---|---|---|---|
| 4 μM LE540) | | Peptide | EC₅₀ [nM] |
| 22R-Hydroxycholesterol | agonistic | TIF2(628-658) | 154 |
| 25-Hydroxycholesterol | agonistic | TIF2(628-658) | 363 |
| (25R)-26-Hydro)qcholesterol | agonistic | TIF2(628-658) | 146 |
| Cholenic Acid Methyl Ester | agonistic | TIF2(628-658) | 311 |
| DMHCA | agonistic | TIF2(628-658) | 85 |

In this variation of the TR-FRET assay, the oxysterol-type compounds do displace an antagonistic compound at saturating concentration (4 μM) in a dose dependent way with calculated EC₅₀ values ranging from 85 nM for DMHCA and 363 nM for 25-Hydroxycholesterol using TIF2 as interacting peptide.

### Example 3

### Radioligand Displacement Assay

This method measures the ability of putative ligands to displace a radioactively labeled compound that is bound to the RORgamma ligand binding domain.

The ligand binding domain (LBD) of RORγ was expressed as fusion protein with GST in BL-21 cells using the vector pDEST15. Cells were lysed by lysozyme-treatment and sonication, and the fusion proteins purified over glutathione sepharose (Pharmacia) according to the manufacturers instructions. For screening of compounds for their ability to bind to RORγ, commercially available 25-26,27-³N-hydroxycholesterol

(Perkin Elmer, NET674250UC) was bound to the protein and displacement by non radioactive ligands was observed. The assay was done in a final volume of 100 μl in a 96-well glutathione and scintillant coated microplate (Perkin Elmer, SMP109001PK). 50-200 ng/well recombinantly expressed RORγ-LBD fused to GST was incubated with 100 nM 25-[26,27-³H]-hydroxycholesterol and 400 nM 25-hydroxycholesterol in a Tris-based buffer (20 mM Tris-HCl pH 7.9; 60 mM KCl, 5 mM MgCl₂; 45 ng/μl BSA). Compounds to be tested were titrated and added to the protein-radioligand mix. DMSO content of the samples was kept at 1%. After addition of the compounds, the assay was equilibrated for 30 min at room temperature. After this incubation the assay plate wells were washed twice with Tris buffer (20 mM Tris-HCl pH 7.5; 60 mM KCl, 5 mM MgCl₂) and subsequently measured for 500 sec per well in a LUMIstar OPTIMA (BMG).

The results of the assay are shown in FIG. 1. The retinoid like structures, LE540, TTNBP and Ch55 but also the oxysterol 25-hydroxycholesterol are, in a dose dependent fashion, able to displace 25-[26,27-³H]-hydroxycholesterol which was prebound to RORgamma ligand binding domain. The apparent potency and efficacy of displacement are highest with LE540 and 25-hydroxycholesterol. TTNBP is less potent but seems rather efficient while Ch55 shows the lowest potency and efficacy in this displacement assay allowing to distinguish among RORgamma interacting ligands.

### Example 4

### Peripheral Blood Mononuclear Cell (PBMC) Stimulation and IL-17 Secretion Assay

Cryopreserved peripheral blood human mononuclear cells (PBMCs) were used for the experiments. The cells were thawed in CTL-Anti-Aggregate-Wash™ solution and washed once in CTL Wash™ medium with benzonase. PBMCs, suspended in CTL serum-free test media (CTL-Test™ Medium), were plated into 96-well BD BioCoat Anti-Human CD3 T-Cell activation plate at a total of 1×10⁵ cells/well in triplicate. The cells were incubated with anti-CD28 (2 μg/ml) in the absence or presence of LE540, LE135 and Am580 at different concentrations for 72 h at 37° C. with 5% CO₂. The compounds were added at the time 0. The supernatants were harvested and assayed for IL-17 according to the protocol from the corresponding ELISA kit (Invitrogen). The detection range was 15.6-1,000.0 pg/ml. Data are presented as means±s.d. values.

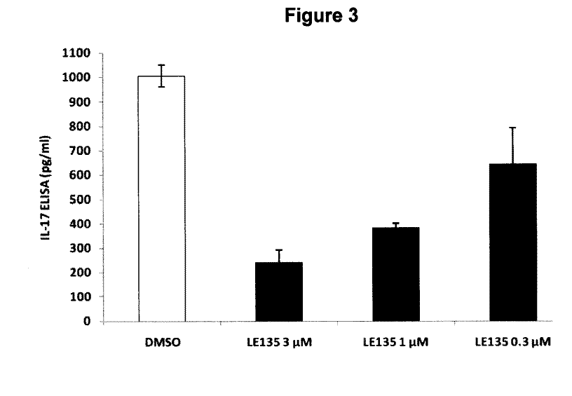

In the control experiments PBMCs were stimulated with anti-CD3 (plate) and anti-CD28 in the presence of 0.1% DMSO. The mean concentration of IL-17 was 1,003.36±45.18 pg/ml. In contrast, the concentration of IL-17 in non stimulated cells was under the detection range (<15.6 pg/ml). LE540, LE135, and Am580 all inhibit the production of IL-17 by human PBMCs. The compounds were added to the cells at time 0 together with the anti-CD28 mAb. The results indicated that, LE540 added at the concentration of 0.3 μM strongly suppressed (mean=289.69 pg/ml; 71.13% of reduction) the production of IL-17. The lower concentration of LE540 (0.1 μM) was also very potent in PBMCs. The percentage of IL-17 inhibition was 34.28% (mean=659.39 pg/ml), respectively. In comparison with these results, LE135 has less inhibitory effect on the production of IL-17 protein in PBMCs. The results presented in FIG. 3 showed that LE135 reduced the level of IL-17 in PBMCs in a dose-response manner. The amount of IL-17 protein was down regulated after treatment of cells with 3, 1, and 0.3 μM of LE135 (FIG. 3). The percentage of inhibition was 75.91, 61.85, and 35.78%, respectively. Incubation of stimulated cells with 10, 5, and 1 μM of Am580 resulted in reduction of IL-17 protein only in a dose group of 10 μM (69.73% of inhibition). In the presence of 5 and 1 μM of Am580 the level of IL-17 was within the control range.

1-15. (canceled)

16. A method for identifying a modulator of RORgamma activity in a biochemical cell-free in vitro assay system comprising the steps of
(a) administrating to such assay system an effective amount of a RORgamma modulator of formula (I), 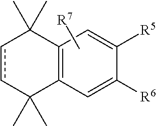
wherein
R⁵ is CONHR⁸, NHCOR⁸, C(O)R⁸, CH═CHR⁸, C(CH₃)═CHR⁸, C═CR⁸, CH(OH)CH═CHR⁸, C(O)CH═CHR⁸, 5 to 6 membered heterocyclyl-R⁸,
R⁶ is hydrogen,
R⁵ and R⁶ may also together form 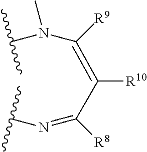
R⁷ is hydrogen, fluorine, chlorine or hydroxy,
R⁸ is 4-yl-benzoic acid or 6-yl-2-naphthoic acid,
R⁹ and R¹⁰ are hydrogen or R⁹ and R¹⁰ form together with the bond to which they attach a fused 5-10 membered heteroaromatic or aromatic monocyclic or bicyclic ring
or an effective amount of the following RORgamma modulators (Z)-4-(10,10,13,13,15-pentamethyl-11,12,13,15-tetrahydro-10H-dinaphtho[2,3-b: 1′,2′-e][1,4]diazepin-7-yl)benzoic acid (LE540), (Z)-4-(5,7,7,10,10-pentamethyl-7,8,9,10-tetrahydro-5H-benzo[e]naphtho[2,3-b][1,4]diazepin-13-yl)benzoic acid (LE135), (E)-4-(2-(5,5,8,8-tetramethyl-5,6,7,8-tetrahydronaphthalen-2-yl)prop-1-enyl)benzoic acid (TTNBP), (E)-4-(3-(3,5-di-tert-butylphenyl)-3-oxoprop-1-enyl)benzoic acid (Ch55), wherein the effective amount is sufficient to induce or reduce the readout of RORgamma activity in such cell culture or biochemical assay system and
(b) comparing the measured activity with the activity of a reference RORgamma modulator.

17. The method of claim 16, wherein the biochemical assay system is selected from a homogenous time-resolved fluorescence resonance energy transfer (HTR-FRET) assay, a radioligand binding and a displacement assay in conjunction with a recombinantly expressed RORgamma protein of the sequence SEQ 1. and a coactivator peptide with a sequence of SEQ 2. or 3.

18. The method of claim 16, wherein the reference RORgamma modulator is one or more of the compounds and is used as a control to monitor the activity of newly to be identified modulators.

19. The method of claim 17, wherein the reference RORgamma modulator is one or more of the compounds and is used as a control to monitor the activity of newly to be identified modulators.


SEQUENCE LISTING (3 sequences)

SEQ ID NO: 1 - protein, 260 aa, Homo sapiens
  RORgamma protein sequence used in FRET and M1H assay types representing aa259-518 of RORgamma (NP_005051.2)
TPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 2 - protein, 25 aa, Artificial
  SRC1(676-700) peptide
CPSSHSSLTERHKILHRLLQEGSPS

SEQ ID NO: 3 - protein, 24 aa, Artificial
  TIF2(628-658) peptide
GQSRLHDSKGQTKLLQLLTTKSDQ